(19) United States
(12) Patent Application Publication
(10) Pub. No.: US 2022/0151985 A1
(43) Pub. Date: May 19, 2022

(54) IMMUNOSTIMULATOR AND FOOD OR BEVERAGE FOR IMMUNOSTIMULATION

(71) Applicant: AMINO UP CO., LTD., Sapporo-shi, Hokkaido (JP)

(72) Inventors: Keima ABE, Sapporo-shi, Hokkaido (JP); Hiroshi NISHIOKA, Sapporo-shi, Hokkaido (JP); Sho TAKANO, Sapporo-shi, Hokkaido (JP); Makiko TAKAHASHI, Sapporo-shi, Hokkaido (JP); Hideyuki MATSUURA, Sapporo-shi, Hokkaido (JP)

(21) Appl. No.: 17/599,311

(22) PCT Filed: Mar. 26, 2020
(86) PCT No.: PCT/JP2020/013504

§ 371 Date: Sep. 28, 2021

(30) Foreign Application Priority Data
Mar. 29, 2019 (JP) 2019-067663

(51) Int. Cl. A61K 31/357; A61K 9/00; A61P 37/04

(52) U.S. Cl. CPC A61K 31/357; A61P 37/04; A61K 9/0053

(57) ABSTRACT

An immunostimulator that comprises a compound represented by general formula I (wherein n stands for an integer of 4-12) or a pharmacologically acceptable salt thereof as an active ingredient.

### TECHNICAL FIELD

The present invention relates to an immunostimulator and a food or beverage for immunostimulation.

### BACKGROUND ART

Up to the present, various substances have been used as immunostimulators for enhancing the immune system. For example, Non Patent Literature 1 states that fucoidan has an immunostimulatory action.

Meanwhile, various physiological activities of carboxylate esters have been reported. For example, Patent Literature 1 discloses an antileishmania activity, and Patent Literature 2 discloses a pathogen controlling activity by plant pathogenic fungi.

### CITATION LIST

### Patent Literature

Patent Literature 1: Japanese Patent Laid-Open No. 2016-160237
Patent Literature 2: Japanese Patent Laid-Open No. 2013-180995

### Non Patent Literature

Non Patent Literature 1: Immune Efficacy and Safety of Fucoidan Extracted from Gagome Kombu® (Kjellmaniella crassifolia®) in Healthy Japanese Subjects, Hiromu Onogi et al., Japanese Journal of Complementary and Alternative Medicine, Vol. 12, Issue 2, 2015, p. 87-93

### SUMMARY OF INVENTION

### Technical Problem

Development of a new immunostimulator has been long-awaited.

An object of the present invention is to provide a novel immunostimulator and a food or beverage for immunostimulation having an excellent immunostimulatory action.

### Solution to Problem

The present inventors have found that a certain compound has an excellent immunostimulatory action, whereby the present invention is accomplished.

For achieving the above object, the immunostimulator according to the first aspect of the present invention comprises, as an active ingredient, a compound represented by general formula I:

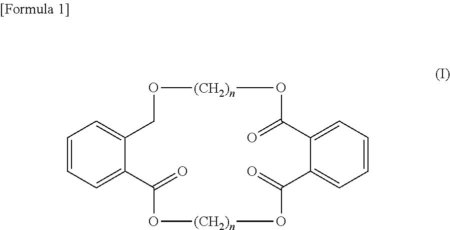

wherein n represents an integer of 4 to 12, or a pharmacologically acceptable salt thereof.

For example, the immunostimulator comprises, as an active ingredient, a compound represented by formula II:

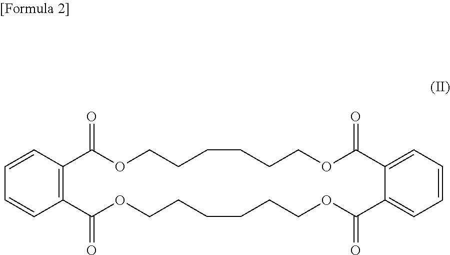 [SMILES: O=C1OCCCCCCOC(=O)c2ccccc2C(=O)OCCCCCCOC(=O)c2ccccc21]

or a pharmacologically acceptable salt thereof.

The food or beverage for immunostimulation according to the second aspect of the present invention comprises, as an active ingredient, a compound represented by general formula I:

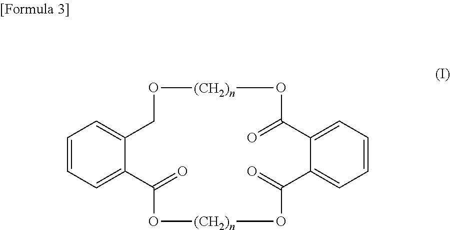

wherein n represents an integer of 4 to 12, or a pharmacologically acceptable salt thereof.

For example, the food or beverage for immunostimulation comprises, as an active ingredient, a compound represented by formula II:

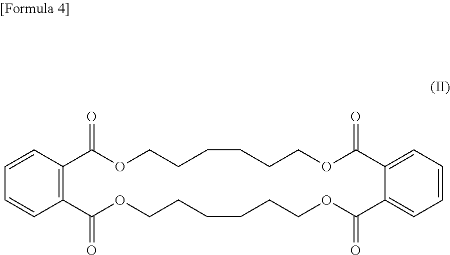 [SMILES: O=C1OCCCCCCOC(=O)c2ccccc2C(=O)OCCCCCCOC(=O)c2ccccc21]

or a pharmacologically acceptable salt thereof.

### Advantageous Effects of Invention

According to the present invention, a novel immunostimulator and a food or beverage for immunostimulation having an excellent immunostimulatory action can be provided.

### BRIEF DESCRIPTION OF DRAWINGS

FIG. 1 shows a method for synthesizing AU-1833C.

FIG. 2 shows ¹³C NMR and ¹H NMR data of the synthesized AU-1833C.

FIG. 3 is a graph showing TNF-α production inducing activities of AU-1833C, Lentinan and Fucoidan.

FIG. 4 shows an immunostimulatory action of AU-1833C, wherein (a) is a graph showing PEC counts, (b) is a graph showing the amount of induced TNF-α production (pg/cell), and (c) is a graph showing the amount of induced TNF-α production (pg/mouse).

### DESCRIPTION OF EMBODIMENTS

First, the immunostimulator of the present embodiment will be described in detail.

The immunostimulator according to the present embodiment comprises, as an active ingredient, a compound represented by general formula I:

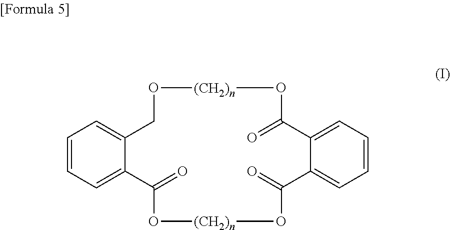

wherein n represents an integer of 4 to 12, or a pharmacologically acceptable salt thereof. In the formula, preferably n is 4 to 10, more preferably n is 4 to 8, and further preferably n is 5 to 7.

As used herein, the term “pharmacologically acceptable salt” means a derivative of the disclosed compound, and in that context, the parent compound is modified by an exchange at an acid or base moiety to be a salt. Examples of the pharmacologically acceptable salt include but are not limited to a mineral or organic acid salt of a basic residue such as amine and alkali, and an organic salt of an acid residue such as a carboxylic acid. The pharmacologically acceptable salt herein includes, for example, a conventional non-toxic salt of a parent compound formed from a non-toxic inorganic or organic acid. Herein, the pharmacologically acceptable salt may be synthesized from a parent compound including a base or acid moiety by a conventional chemical method. Typically, such a salt may be prepared by reacting a free acid or free base of the compound with a stoichiometric amount of an appropriate base or acid in water or an organic solvent or in a mixed solution thereof. Generally, non-aqueous media such as ether, ethyl acetate (EtOAc), ethanol, isopropanol, and acetonitrile are preferable. A list of reasonable salts is shown in Remington's Pharmaceutical Sciences, 17th ed., Mack Publishing Company, Easton, Pa., 1985, p. 1418 and Journal of Pharmaceutical Science, 66, 2 (1977).

The compound of the general formula I set forth above can be produced by, for example, a known synthesis method such as “Synthesis of New Macrocycles. Part IV. I Two-step Synthesis of Dimeric Phthalic Acid Esters. Journal of the Chemical Society, Perkin Transactions 1, 1974, 2578-2580.”

The immunostimulator according to the present embodiment may comprise, as an active ingredient, a compound represented by formula II:

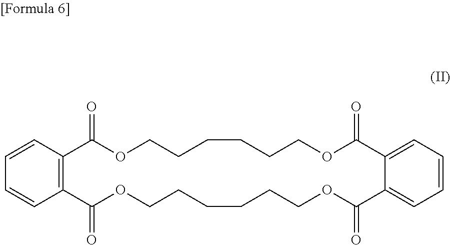 [SMILES: O=C1OCCCCCCOC(=O)c2ccccc2C(=O)OCCCCCCOC(=O)c2ccccc21]

or a pharmacologically acceptable salt thereof. The compound of formula II is a compound wherein n is 6 in the formula I described above. The chemical name for the compound of formula II is 7,8,9,10,11,12,19,20,21,22,23,24-dodecahydro-dibenzo[1,6,13,18]-tetraoxacyclooctadecine-2,5,14,17-tetrone. Herein, the compound of formula II is sometimes referred to as “AU-1833C.”

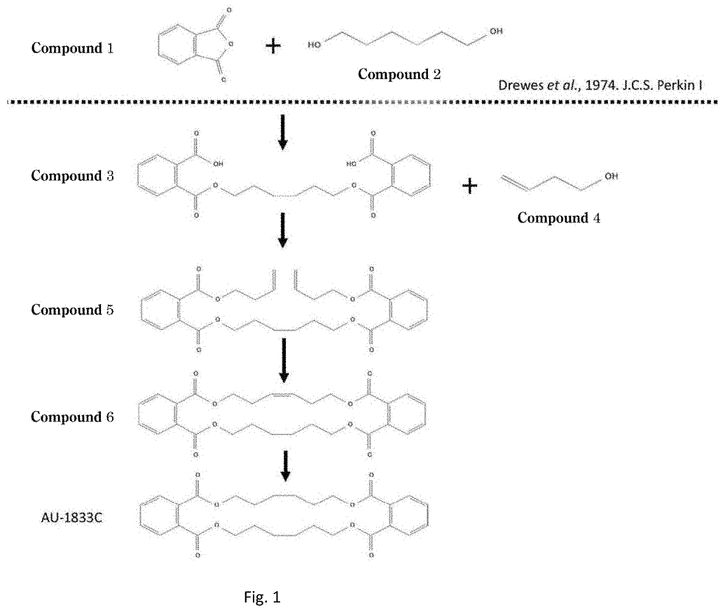

An example of the method for synthesizing the compound of formula II (AU-1833C) set forth above will be described (FIG. 1). Hereinbelow, Compounds 1 to 5 are as shown in FIG. 1. Compound 1 is dissolved in pyridine, Compound 2 is added thereto and the resultant is stirred for a specified time at a specified temperature. Compound 1 is further added and the resultant is stirred for a specified time at a specified temperature. Then, after dilution with EtOAc, an HCl solution, a saturated sodium hydrogen carbonate solution, and a saturated sodium chloride solution are added in this order for separation. The EtOAc layer is dried by adding Na₂SO₄ and then concentrated to obtain Compound 3. Compound 3, 4-dimethylaminopyridine (DMAP), and N,N′-dicyclohexylcarbodiimide (DCC) are dissolved in anhydrous dichloromethane, and Compound 4 is added thereto and the resultant is stirred for a specified time. Next, after dilution with chloroform, an HCl solution, a saturated sodium hydrogen carbonate solution, and a saturated sodium chloride solution are added in this order for separation. The chloroform layer is dried by adding Na₂SO₄, then concentrated, and purified using a silica gel column with EtOAc and hexane thereby to obtain Compound 5. Compound 5 and a specified catalyst are dissolved in anhydrous dichloromethane and stirred for a specified time. Next, purification is carried out using a silica gel column with EtOAc and hexane thereby to obtain Compound 6. Compound 6 is dissolved in methanol, a palladium-activated carbon ethylenediamine complex is added thereto and the resultant is stirred for a specified time in a hydrogen atmosphere. Next, after celite filtration, purification is carried out using a silica gel column with EtOAc and hexane thereby to obtain AU-1833C. Note that the compound of formula II (AU-1833C) set forth above can be synthesized by a method other than this, or can be isolated by processing, extracting and the like of an animal or a plant.

The immunostimulatory action of the immunostimulator according to the present embodiment can be determined to be present, when, for example, the degree of cytokine production enhancement (for example, tumor necrosis factor (TNF-α) production enhancement) in mammalian cells to which the immunostimulator is administered is higher than that in non-administered mammalian cells, or the degree of an increase in the peritoneal exudate cell (PEC) count in a mammal to which the immunostimulator is administered is higher than that in a non-administered mammal. Examples of the mammal mentioned above include humans, mice, monkeys, horses, and cows.

The immunostimulator according to the present embodiment can be prepared by a common method into a dosage form such as a tablet, a granule, a powder, a capsule, a syrup, and an injection, and an appropriate drug delivery system (DDS) can also be used. Additionally, an excipient, a binder, a lubricant, a colorant, a disintegrator, a thickener, a preservative, a stabilizer, and a pH adjusting agent typically used in pharmaceutical products can be added. Additionally, the dose of the immunostimulator according to the present embodiment can be suitably determined in accordance with the age, body weight, symptoms to be applied, and the like of a subject. Additionally, the administration method of the immunostimulator according to the present embodiment can be any of administrations with a meal, after a meal, before a meal, between meals, and at bedtime.

Next, the food or beverage for immunostimulation according to the present embodiment will be described.

The food or beverage for immunostimulation according to the present embodiment comprises, as an active ingredient, a compound represented by general formula I:

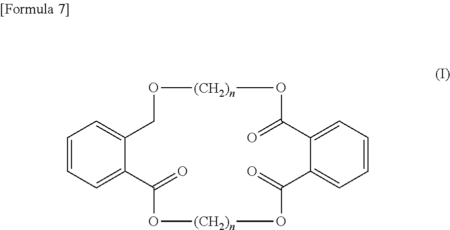

wherein n represents an integer of 4 to 12, or a pharmacologically acceptable salt thereof. In the formula, preferably n is 4 to 10, more preferably n is 4 to 8, and further preferably n is 5 to 7.

The food or beverage for immunostimulation according to the present embodiment can comprise, as an active ingredient, for example, a compound represented by formula II:

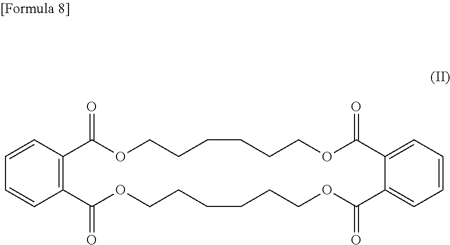 [SMILES: O=C1OCCCCCCOC(=O)c2ccccc2C(=O)OCCCCCCOC(=O)c2ccccc21]

or a pharmacologically acceptable salt thereof. The compound of formula II is a compound wherein n is 6 in formula I set forth above. The chemical name for the compound of the formula II is 7,8,9,10,11,12,19,20,21,22,23,24-dodecahydro-dibenzo[1,6,13,18]-tetraoxacyclooctadecine-2,5,14,17-tetrone. Herein, the compound of formula II is sometimes referred to as “AU-1833C.”

For the food or beverage for immunostimulation according to the present embodiment, details of the “pharmacologically acceptable salt” and “immunostimulatory action” are the same as described above.

The food or beverage for immunostimulation according to the present embodiment can be processed by a common method into a form suitable for eating and drinking such as a granular form, a grain form, a tablet, a capsule, a gel form, a cream form, a paste form, a suspension form, an aqueous solution form, an emulsion form, and a powder form. Additionally, an excipient, a binder, a lubricant, a colorant, a disintegrator, a thickener, a preservative, a stabilizer, and a pH adjusting agent typically used in foods and drinks can be added. Further, for the improvement of taste qualities, saccharides, sugar alcohols, salts, fats and oils, amino acids, organic acids, and glycerin can be added in the range not affecting the effects of the present invention. Note that when the food or beverage for immunostimulation according to the present embodiment contained in an existing food or drink is used, the food or drink to be the base can be suitably selected as long as such a food or drink can provide the effects of the present invention.

The food or beverage for immunostimulation according to the present embodiment can be used as, for example, a food for specified health use, a food with nutrient function claims, a food with functional claims, a supplement, a so-called energy drink, livestock feed and the like.

### EXAMPLES

Hereinafter, the present invention will specifically be described in reference to Examples. However, the present invention is not limited to these Examples.

### Example 1

As shown in FIG. 1, the compound of formula II (AU-1833C) was synthesized. Compounds 1 to 6 are as shown in FIG. 1.

### (Synthesis of Compound 5)

3 g (20 mmol) of Compound 1 (manufactured by FUJIFILM Wako Pure Chemical Corporation) was dissolved in 40 mL of pyridine (manufactured by FUJIFILM Wako Pure Chemical Corporation), 1.9 g (16 mmol) of Compound 2 (manufactured by FUJIFILM Wako Pure Chemical Corporation) was added thereto and the resultant was stirred at 80° C. for 4 hours. Further, 3 g (20 mmol) of Compound 1 was added and the resultant was stirred at 80° C. for 20 hours. Next, after dilution with EtOAc, 1 M of an HCl solution, a saturated sodium hydrogen carbonate solution, and a saturated sodium chloride solution were added in this order for separation. The EtOAc layer was dried by adding Na₂SO₄ and then concentrated to obtain Compound 3 (5 g, 12 mmol, yield 63%). Compound 3 (5 g, 12 mmol), 1.2 g (1 mmol) of 4-dimethylaminopyridine (DMAP; manufactured by FUJIFILM Wako Pure Chemical Corporation), and 10.3 g (5 mmol) of N,N′-dicyclohexylcarbodiimide (DCC; manufactured by FUJIFILM Wako Pure Chemical Corporation) were dissolved in anhydrous dichloromethane (70 mL), 2.2 g (30 mmol) of Compound 4 (manufactured by FUJIFILM Wako Pure Chemical Corporation) was added thereto and the resultant was stirred overnight. Next, after dilution with chloroform, 1 M of an HCl solution, a saturated sodium hydrogen carbonate solution, and a saturated sodium chloride solution were added in this order for separation. The chloroform layer was dried by adding Na₂SO₄, then concentrated and purified using a silica gel column (200 g) with EtOAc:hexane=40:60 (v/v) thereby to obtain Compound 5 (3.5 g, 6.7 mmol, yield from Compound 3 was 56%). The ¹H-NMR analysis result for Compound 5 is shown below; ¹H-NMR (CDCl₃, 270 MHz) δ 7.68 (4H, dd, J=5.7, 3.5 Hz), δ 7.49 (4H, dd, J=5.4, 3.2 Hz), δ 5.80 (2H, m), δ 5.10 (4H, m), δ 4.30 (8H, m), δ 2.46 (4H, m), δ 1.73 (4H, m), δ 1.49 (4H, m).

### (Synthesis of Compound 6)

Compound 5 (3.5 g, 6.7 mmol) and 150 mg (177 mmol) of Grubbs catalyst 2nd generation (manufactured by Sigma-Aldrich Co. LLC) were dissolved in anhydrous dichloromethane (100 mL) and the resultant was stirred overnight. Next, purification was carried out using a silica gel column (200 g) with EtOAc:hexane=35:65 (v/v) thereby to obtain Compound 6 (1.3 g, 2.6 mmol, yield from Compound 5 was 39%). The ¹H-NMR analysis result for Compound 6 is shown below; ¹H NMR (CDCl₃, 270 MHz) δ 7.72 (4H, m), δ 7.53 (4H, m), δ 5.59 (2H, m), δ 4.30 (8H, m), 02.46 (4H, m), δ 1.75 (4H, m), δ 1.46 (4H, m).

### (Synthesis of AU-1833C)

Compound 6 (1.3 g, 2.6 mmol) was dissolved in methanol (60 mL), 60 mg of a palladium-activated carbon ethylenediamine complex (manufactured by FUJIFILM Wako Pure Chemical Corporation) was added thereto and the resultant was stirred for 3 hours in a hydrogen atmosphere. Next, after celite filtration, purification was carried out using a silica gel column (100 g) with EtOAc:hexane=40:60 (v/v) thereby to obtain AU-1833C (0.9 g, 1.8 mmol, yield from Compound 6 was 68%.)

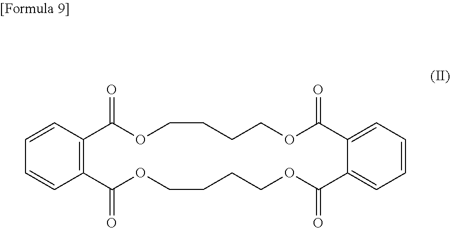 [SMILES: O=C1OCCCCOC(=O)c2ccccc2C(=O)OCCCCOC(=O)c2ccccc21]

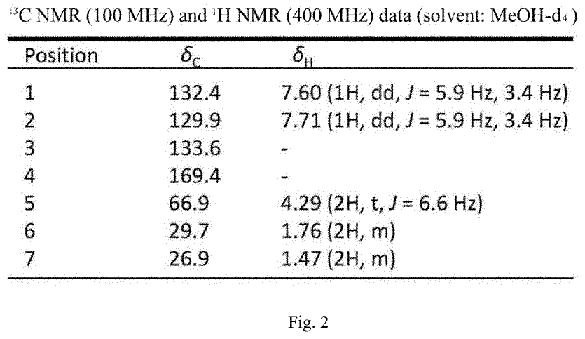

¹³C NMR (100 MHz) data and ¹H NMR (400 MHz) data of the synthesized compound are shown in FIG. 2 (solvent: MeOH-d₄). These data revealed that the synthesized compound was definitely the compound of formula (II) (AU-1833C).

### Example 2

An immunostimulatory action of AU-1833C was investigated.

### (TNF-α Production-Inducing Activity in Cells)

A TNF-α production-inducing activity in a mouse-derived macrophage-like cell line (RAW264, provided: RIKEN BioResource Research Center) was investigated. Using, as test samples, AU-1833C (synthesized in Example 1), lentinan (product name: Lentinan, manufactured by FUJIFILM Wako Pure Chemical Corporation) and fucoidan (product name: Fucoidan Fucus vesiculosus-derived, manufactured by Sigma-Aldrich Co. LLC), test sample solutions were prepared so that the final concentrations of the test samples were 50, 100, and 200 μg/mL. First, all test samples were dissolved in dimethylsulfoxide (DMSO) so as to have a 1,000-fold concentration of the final concentration, and then 100-fold dilution was carried out with deionized water thereby to prepare test sample solutions having a 10-fold concentration (including 1% DMSO) of the final concentrations. RAW264 cells were suspended in DMEM medium (product name: Dulbecco's Modified Eagle Medium “Nissui” 2 powder, manufactured by NISSUI PHARMACEUTICAL CO., LTD.) to which 10% fetal bovine serum (FBS) was added, seeded in a 96-well microplate in a cell density of 10,000 cells/well/100 μL, and cultured at 37° C. for 16 hours under 5% CO₂. The medium was replaced with fresh DMEM medium (180 μL), and the test sample solution in an amount of one tenth (20 μL) as each test sample group or a 1% DMSO aqueous solution as a non-treated group was added and the cells were cultured at 37° C. for 24 hours under 5% CO₂. Thereafter, the culture supernatant was collected and subjected to an evaluation test of the TNF-α production-inducing activity. The concentration measurement of the produced TNF-α was carried out using an ELISA kit (product name: Mouse TNF-alpha Quantikine ELISA Kit, manufactured by R&D systems) in accordance with protocol. A ratio of a TNF-α concentration in each test sample group relative to a TNF-α concentration in the non-treated group was calculated from the measured TNF-α concentrations and defined as the TNF-α production-inducing activity.

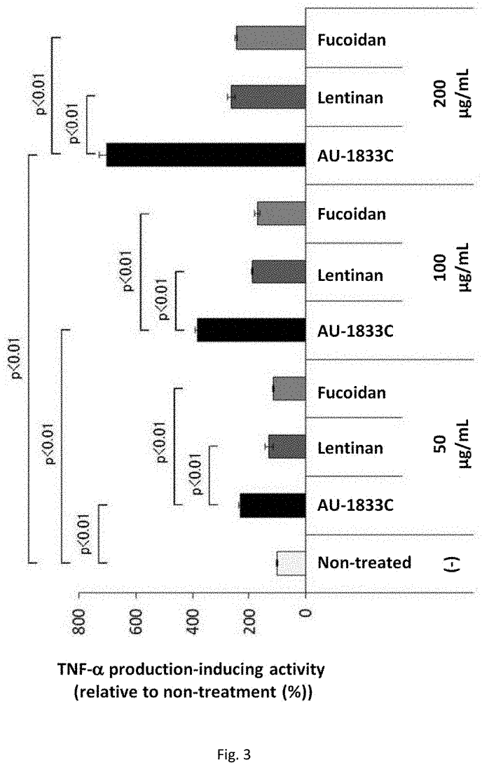

The results of TNF-α production-inducing activity are shown in FIG. 3. At all concentrations of 50, 100, and 200 μg/mL, the TNF-α production-inducing activity of AU-1833C was revealed to be significantly higher than that of the control non-treated group. Additionally, the TNF-α production-inducing activity of AU-1833C at each concentration was revealed to be significantly higher than those of lentinan and fucoidan at the same concentration.

The above revealed that AU-1833C had a significantly higher TNF-α production-inducing activity than lentinan and fucoidan which are the existing immunostimulators, thereby revealing that AU-1833C had an excellent immunostimulatory action.

### (PEC Count and the Amount of Induced TNF-α Production in Mice)

Next, peritoneal exudate cell (PEC) counts and the amount of induced TNF-α production in mice were investigated.

The test sample AU-1833C was dissolved in DMSO so as to be 50 mg/mL and then diluted five-fold with saline (product name: Otsuka normal saline, manufactured by Otsuka Pharmaceutical Co., Ltd.) to prepare a test sample solution of 10 mg/mL (including 20% DMSO).

The animals used were Slc: ddY mice (female, 6-week old) and grouped as follows.

1) Non-treated group: n=6

2) AU-1833C group: n=6

The mice purchased at the age of 5 weeks were acclimated for 5 days under a condition of free access to water and feed (regular diet (product name: CE-2, manufactured by CLEA Japan, Inc.)). Body weights of the mice after acclimation at the age of 6 weeks were measured and the mice were grouped based on the measured values so that there was no difference in the body weight among the groups. 200 μL of the test sample solutions or 20% DMSO saline as the non-treated group was respectively administered to the mice by intraperitoneal injection. The mice after a lapse of 22 hours from administration were sacrificed under isoflurane anesthesia, 5 mL of saline was intraperitoneally injected, the abdominal area was rubbed about 10 times, and then intraperitoneal cells were collected by collecting the saline from the abdominal cavity. Using a portion of the collected saline and a Turk's solution (product name: Turk's solution, manufactured by Wako Pure Chemical Industries, Ltd.), a concentration of PEC (cells/mL), intraperitoneally exudated nucleated cells such as white blood cells, was measured. The measured PEC concentration was multiplied by the amount of saline intraperitoneally injected (5 mL) to calculate a PEC count per mouse (cells/mouse).

The remaining of the collected saline was centrifuged to collect PECs for measuring the TNF-α production-inducing ability of PEC when treated with lipopolysaccharide (LPS) (product name: lipopolysaccharide from Escherichia coli O111: B4, manufactured by Sigma-Aldrich Co. LLC). PECs were suspended in serum free RPMI 1640 medium (product name: RPMI 1640 medium “Nissui” 2 powder, manufactured by NISSUI PHARMACEUTICAL CO., LTD.), and then washed by centrifugation again and suspended in RPMI 1640 medium to which 10% fetal bovine serum (FBS) was added. Using a Turk's solution, the cell count after wash was calculated again, then the concentration was adjusted with serum-containing RPMI 1640, and the cells were seeded in a 96-well microplate in a cell density of 800,000 cells/well/180 μL. LPS was added to make a total medium volume of 200 μL in such a way that the final concentration was 100 ng/mL, and the cells were cultured for 3 hours at 37° C. under 5% CO₂. Thereafter, the culture supernatant was collected and subjected to an evaluation test of the amount of induced TNF-α production. The measurement of a TNF-α concentration was carried out using an ELISA kit in accordance with the protocol. From the measured TNF-α concentration (pg/1,000 μL), a TNF-α amount per cell was calculated using the following equation 1, and a TNF-α amount produced by PEC per mouse was calculated using the following equation 2.

$$\begin{matrix}TNF - α concentration ( pg / 1 , 000 µL ) \times ( 200 µL / 1 , 000 ) / 800 , 000 cells = TNF - α production amount ( pg / cell ) & Equation 1 \\ TNF - α concent ration ( pg / 1 , 000 µL ) \times ( 200 µL / 1 , 000 ) / 800 , 000 cells \times P EC count per mouse ( cells / mouse ) = TNF - α production amount ( pg / mouse ) & Equation 2\end{matrix}$$

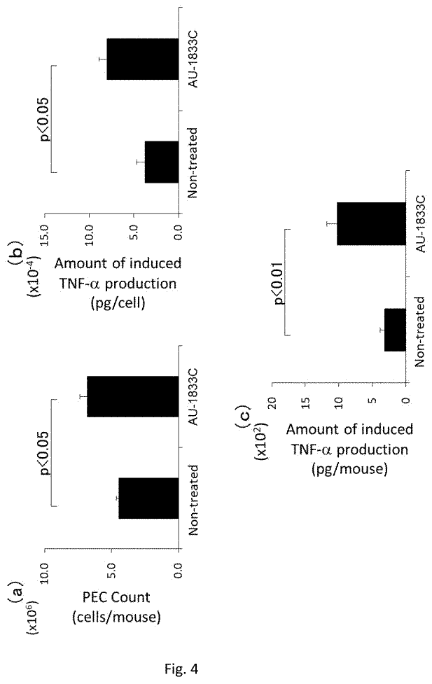

The results are shown in FIG. 4. It was revealed that the mice to which AU-1833C was intraperitoneally administered had significantly higher PEC counts (FIG. 4(a)) as compared with the control non-treated mice. Additionally, it was revealed that the mice to which AU-1833C was administered had a significantly higher TNF-α production amount per cell in PEC (FIG. 4(b)) and also had a significantly higher PEC-derived TNF-α production amount per mouse (FIG. 4(c)) than the control non-treated mice.

The above revealed that the AU-1833C administration increased the PEC count, increased the amount of induced TNF-α production in PECs, and increased the amount of PEC-derived induced TNF-α production per mouse, thereby revealing that AU-1833C had an excellent immunostimulatory action.

Note that the present invention can be carried out in various embodiments and modifications without departing from the broad spirit and scope of the present invention. Additionally, the embodiments described above are for the purpose of describing the present invention and do not intend to limit the scope of the present invention. That is, the scope of the present invention is defined by the claims, but not by the embodiments. Thus, various modifications carried out within the claims and within the scope of meaning of the invention equivalent thereto are considered within the scope of the present invention.

The present invention is based on Japanese Patent Application No. 2019-67663 filed on Mar. 29, 2019. The description, the claims, and all the drawings disclosed in Japanese Patent Application No. 2019-67663 shall be incorporated herein by reference in its entirety.

1. A method for inducing immunostimulatory action comprising administering a compound represented by general formula I: 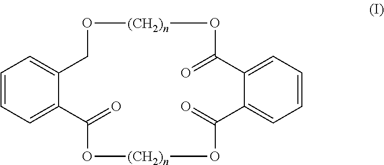
wherein n represents an integer of 4 to 12, or a pharmacologically acceptable salt thereof.

2. The method according to claim 1, comprising administering a compound represented by formula II: 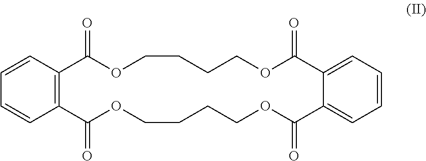 [SMILES: O=C1OCCCCOC(=O)c2ccccc2C(=O)OCCCCOC(=O)c2ccccc21]
or a pharmacologically acceptable salt thereof.

3. A food or beverage for immunostimulation comprising, as an active ingredient, a compound represented by general formula I: 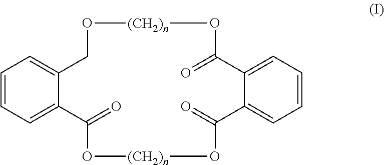
wherein n represents an integer of 4 to 12, or a pharmacologically acceptable salt thereof.

4. The food or beverage for immunostimulation according to claim 3 comprising, as an active ingredient, a compound represented by formula II: 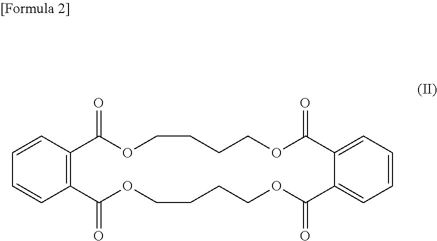 [SMILES: O=C1OCCCCOC(=O)c2ccccc2C(=O)OCCCCOC(=O)c2ccccc21]
or a pharmacologically acceptable salt thereof.

5. A pharmaceutical composition for immunostimulation comprising, as an active ingredient, a compound represented by general formula I: 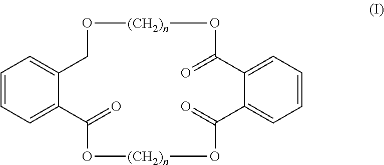
wherein n represents an integer of 4 to 12, or a pharmacologically acceptable salt thereof.

6. The pharmaceutical composition for immunostimulation according to claim 5 comprising, as an active ingredient, a compound represented by formula II: 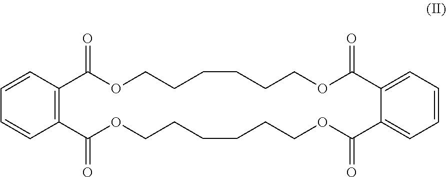 [SMILES: O=C1OCCCCCCOC(=O)c2ccccc2C(=O)OCCCCCCOC(=O)c2ccccc21]
or a pharmacologically acceptable salt thereof.
